(19) United States
(12) Patent Application Publication
(10) Pub. No.: US 2006/0282139 A1
(43) Pub. Date: Dec. 14, 2006

(54) DEVICES AND METHODOLOGIES USEFUL IN NON INVASIVE TERMINATION OF PREGNANCY

(75) Inventor: David Weintraub, Yavne (IL)

Correspondence Address: DARBY & DARBY P.C., P. O. BOX 5257, NEW YORK, NY, 10150-5257, US

(73) Assignee: ULTRASTOP LTD., Tel Aviv (IL)

(21) Appl. No.: 11/466,261

(22) Filed: Aug. 22, 2006

Related U.S. Application Data
(62) Division of application No. 10/872,072, filed on Jun. 18, 2004.

(51) Int. Cl. A61F 7/00; A61F 7/12

(52) U.S. Cl. 607/96

(57) ABSTRACT

A method and apparatus for termination of pregnancy, the method including imaging a gestational sac in a body and applying energy through non-gestational sac body tissue to the gestational sac, which is sufficient to effect termination of pregnancy and the apparatus including a radiant energy source and a gestational sac irradiator, adapted to be operative to irradiate a gestational sac through non-gestational sac body tissue with sufficient energy from the radiant energy source so as to cause termination of pregnancy.

### FIELD OF THE INVENTION

The present invention relates to non-invasive termination of pregnancy.

### BACKGROUND OF THE INVENTION

The following U.S. Patents are believed to represent the current state of the art:

U.S. Pat. Nos. 5,356,876; 4,609,552; 4,780,312 and 4,073,899.

### SUMMARY OF THE INVENTION

The present invention seeks to provide improved apparatus and methodology for termination of pregnancy from outside of the body.

There is thus provided in accordance with a preferred embodiment of the present invention a method for termination of pregnancy including imaging a gestational sac in a body and applying energy through non-gestational sac body tissue to the gestational sac, which is sufficient to effect termination of pregnancy.

In accordance with a preferred embodiment of the present invention the imaging includes ultrasound imaging. Alternatively, the imaging includes MRI imaging. In another preferred embodiment of the present invention the imaging includes CT imaging.

In accordance with a preferred embodiment of the present invention the energy includes ultrasound energy. Alternatively, the energy includes electromagnetic energy.

In accordance with a preferred embodiment of the present invention the applying produces a thermal effect on the gestational sac. Alternatively, the applying produces a cavitation effect on the gestational sac. In accordance with another preferred embodiment of the present invention the applying produces a micro-streaming effect on the gestational sac. Alternatively, the applying produces a jackhammer effect on the gestational sac.

In accordance with a preferred embodiment of the present invention the imaging is operative to image results of the applying.

In accordance with a preferred embodiment of the present invention the method also includes directing the energy to a target volume at least partially including the gestational sac. Additionally, the directing generally prevents pathological damage to tissue outside of the target volume. Additionally or alternatively, the directing generally focuses the energy on the gestational sac. In accordance with another preferred embodiment of the present invention the directing includes positioning at least one transducer relative to the body. Additionally, the directing includes locating a focus of at least one transducer at the target volume. In accordance with yet another preferred embodiment of the present invention the directing includes varying a location of a focus of at least one transducer at the target volume. In accordance with still another preferred embodiment of the present invention the varying a location of a focus changes a volume of the target volume.

In accordance with a preferred embodiment of the present invention the method also includes obtaining a feedback indication of the applying.

In accordance with another preferred embodiment of the present invention the applying employs a transducer located outside of the body.

In accordance with another preferred embodiment of the present invention, the method includes modulating the energy to effect termination of pregnancy while generally preventing pathological damage to non-gestational sac tissue within the target volume.

In accordance with a preferred embodiment of the present invention the modulating includes modulating the amplitude of the ultrasound energy over time.

In accordance with another preferred embodiment of the present invention the applying employs ultrasound energy in a continuous mode.

There is also provided in accordance with a preferred embodiment of the present invention apparatus for termination of pregnancy including a radiant energy source and a gestational sac irradiator, adapted to be operative to irradiate a gestational sac through non-gestational sac body tissue with sufficient energy from the radiant energy source so as to cause termination of pregnancy.

In accordance with a preferred embodiment of the present invention the apparatus also includes a gestational sac imager.

In accordance with another preferred embodiment of the present invention the apparatus also includes a radiant energy modulator operative to modulate the radiant energy so as to have characteristics which enable termination of pregnancy without substantial pathological effects on non-gestational sac body tissue within an irradiated target volume containing the gestational sac.

In accordance with a preferred embodiment of the present invention the radiant energy is focused energy.

In accordance with another preferred embodiment of the present invention the imager provides ultrasound imaging. Alternatively, the imager provides MRI imaging. In accordance with still another preferred embodiment of the present invention the imager provides CT imaging.

In accordance with a preferred embodiment of the present invention the energy includes ultrasound energy. Alternatively, the energy includes electromagnetic energy.

In accordance with a preferred embodiment of the present invention the gestational sac irradiator produces a thermal effect on the gestational sac. Alternatively, the gestational sac irradiator produces a cavitation effect on the gestational sac. In accordance with another preferred embodiment of the present invention the gestational sac irradiator produces a micro-streaming effect on the gestational sac. Alternatively, the gestational sac irradiator produces a jackhammer effect on the gestational sac.

In accordance with a preferred embodiment of the present invention the imager is operative to image results of operation of the gestational sac irradiator.

In accordance with another preferred embodiment of the present invention the gestational sac irradiator includes an energy director operative to direct the energy to a target volume at least partially including the gestational sac. Additionally, the director generally prevents pathological damage to tissue outside of the target volume. In accordance with a preferred embodiment of the present invention the director generally focuses the energy on the gestational sac. Preferably, the director includes at least one transducer selectably positionable relative to the body. In accordance with still another preferred embodiment of the present invention the director focuses energy from at least one transducer at the target volume. In accordance with yet another preferred embodiment of the present invention the director is operative to vary a location of a focus of at least one transducer at the target volume. Additionally, the director, by varying the location of the focus, changes a volume of the target volume.

In accordance with a preferred embodiment of the present invention the apparatus also includes an irradiation feedback indication functionality.

In accordance with another preferred embodiment of the present invention the transducer is adapted to be located outside of the body.

In accordance with another preferred embodiment of the present invention the apparatus also includes an energy modulator to effect termination of pregnancy while generally preventing pathological damage to non-gestational sac tissue within the target volume.

In accordance with yet another preferred embodiment of the present invention the modulator modulates the amplitude of the ultrasound energy over time.

In accordance with still another preferred embodiment of the present invention the irradiator employs ultrasound energy in a continuous mode.

In accordance with a preferred embodiment of the present invention the ultrasound energy has a frequency in a range of 100 KHz-5000 KHz. In accordance with another preferred embodiment of the present invention, the ultrasound energy has a frequency in a range of 100 KHz-300 KHz. In accordance with yet another preferred embodiment of the present invention, the ultrasound energy has a frequency in a range of 1000 KHz-3000 KHz.

In accordance with a preferred embodiment of the present invention the modulating provides a duty cycle between 1:2 and 1:250. In accordance with another preferred embodiment of the present invention the modulating provides a duty cycle between 1:5 and 1:100. In accordance with yet another preferred embodiment of the present invention the modulating provides a duty cycle between 1:10 and 1:80.

In accordance with a preferred embodiment of the present invention the modulating provides between 2 and 1000 sequential cycles at an amplitude above a cavitation threshold. In accordance with another preferred embodiment of the present invention the modulating provides between 25 and 500 sequential cycles at an amplitude above a cavitation threshold. In accordance with yet another preferred embodiment of the present invention the modulating provides between 100 and 300 sequential cycles at an amplitude above a cavitation threshold.

### BRIEF DESCRIPTION OF THE DRAWINGS

The present invention will be understood and appreciated more fully from the following detailed description, taken in conjunction with the drawings and appendix in which:

FIG. 1 is a simplified pictorial illustration of the general structure and operation of a termination of pregnancy system constructed and operative in accordance with a preferred embodiment of the present invention;

FIG. 2 is a simplified block diagram illustration of a preferred power source and modulator useful in the system of FIG. 1, showing a pattern of variation of ultrasound pressures over time in accordance with a preferred embodiment of the present invention;

FIGS. 3A-3C are illustrations of an operator interface display during operation;

FIG. 4 is a simplified block diagram illustration of the termination of pregnancy treatment system of FIG. 1; and

FIG. 5 is a simplified flowchart illustrating steps in termination of pregnancy in accordance with a preferred embodiment of the present invention.

### DETAILED DESCRIPTION OF PREFERRED EMBODIMENTS

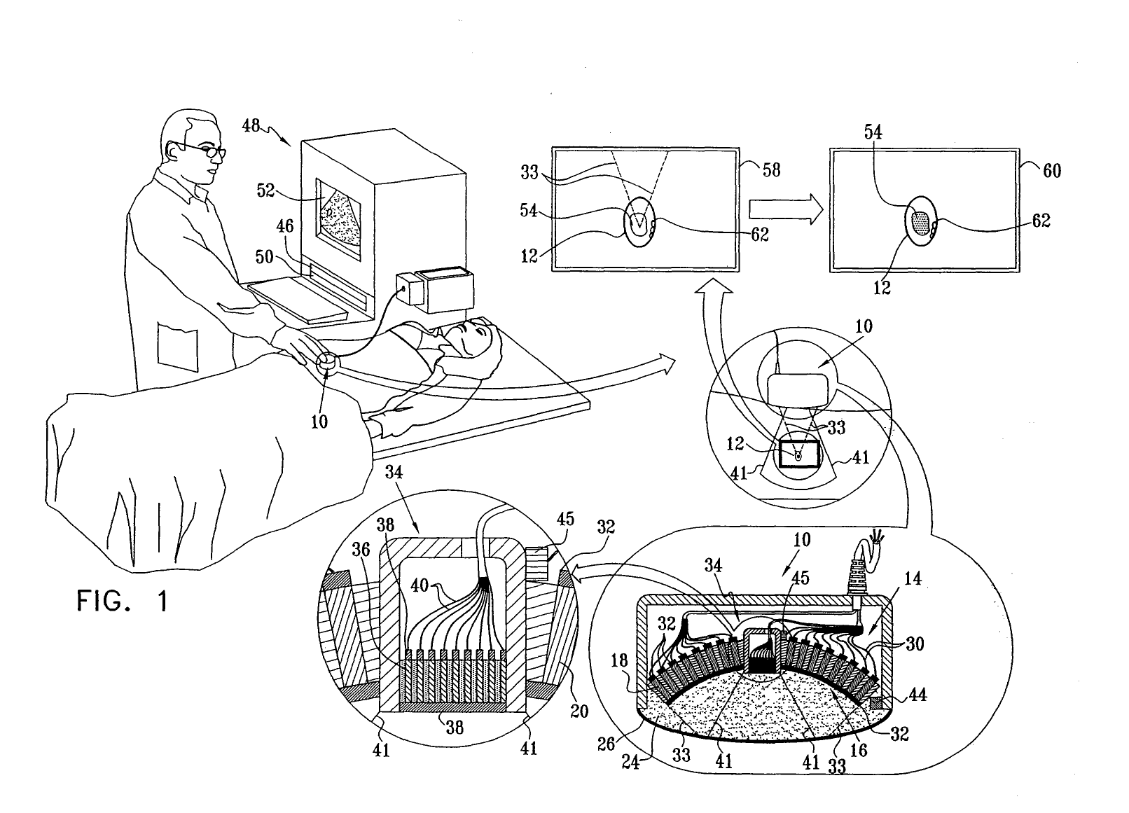

Reference is now made to FIG. 1, which is a simplified pictorial illustration of the general structure and operation of a non-invasive termination of pregnancy system constructed and operative in accordance with a preferred embodiment of the present invention. As seen in FIG. 1, an energy generator and director, such as an ultrasound transducer subsystem 10, disposed outside a body, generates energy which, by suitable placement of the transducer subsystem 10 relative to the body, is directed to a target volume 12 inside the body and is selectively operative to cause energy to impinge thereon, so as to adversely affect the gestational sac, thereby to result in termination of pregnancy, without pathological effects on other tissue.

A preferred embodiment of an ultrasound transducer subsystem 10 comprises an ultrasound therapeutic transducer assembly 14 including a focusing transducer 16, preferably including a curved or planar phased array of transducers 18, typically defining a portion of a sphere. The transducers 18 may be of any suitable configuration, shape and distribution. Preferably, transducers 18 are piezoelectric transducers.

In another preferred embodiment, the energy generator and director may comprise an electromagnetic energy generator and director.

Preferably, transducers 18 are embedded in a vibration damping material 20 to avoid mechanical cross talk between transducers 18. A cooling system (not shown) may be associated with the transducers 18. An intermediate element 24 preferably is formed of a material, such as castor oil or any other suitable fluid, with acoustic impedance similar to that of water and a high cavitation threshold, enclosed by a thin layer of material 26, such as polyurethane, which has acoustic impedance similar to that of soft mammalian tissue, defining a contact surface which may be generally planar but need not be.

Alternatively, the intermediate element 24 may be formed of a material, such as polyurethane, having acoustic impedance similar to that of soft mammalian tissue, and defines a contact surface for engagement with the body, typically via a suitable coupling gel or oil (not shown).

Suitably modulated AC electrical power is supplied by conductors 30 to conductive coatings 32 on piezoelectric transducers 18 to cause the transducers 18 to provide a desired focused acoustic energy output, represented by dashed lines 33.

In accordance with a preferred embodiment of the present invention, an imaging ultrasound transducer subassembly 34 is incorporated within ultrasound therapeutic transducer assembly 14 and typically comprises multiple piezoelectric transducers 36 having conductive surfaces 38 associated with opposite edge surfaces thereof. Alternatively, imaging ultrasound transducer subassembly 34 may be located outside ultrasound therapeutic transducer assembly 14. Suitably modulated AC electrical power is supplied by conductors 40 to conductive surfaces 38 of piezoelectric transducer 36 in order to cause the piezoelectric transducer 36 to provide an acoustic energy output. Conductors 40, coupled to conductive surfaces 38, also provide an imaging output from imaging ultrasound transducer subassembly 34, which is represented by solid lines 41.

It is appreciated that commercially available high frequency ultrasound transducers may be employed for imaging. Alternatively, MRI imaging or CT imaging may be provided.

It is further appreciated that various types of ultrasound transducer subsystems 10 may be employed. For example, such transducer subsystems may include multiple piezoelectric elements, multi-layered piezoelectric elements and piezoelectric elements of various shapes and sizes arranged in a phased array. As a further alternative, the ultrasound transducer subsystem 10 may include a single piezoelectric element.

In a preferred embodiment of the present invention shown in FIG. 1, the ultrasound energy generator and director are combined in transducer assembly 10. Alternatively, the functions of generating ultrasound energy and focusing such energy may be provided by distinct devices.

In accordance with a preferred embodiment of the present invention, a skin temperature sensor 44, such as an infrared sensor, may be mounted in proximity to the contact surface 26 as shown in FIG. 1 Further in accordance with a preferred embodiment of the present invention a transducer temperature sensor 45, such as a thermocouple, may also be mounted alongside imaging ultrasound transducer subassembly 34.

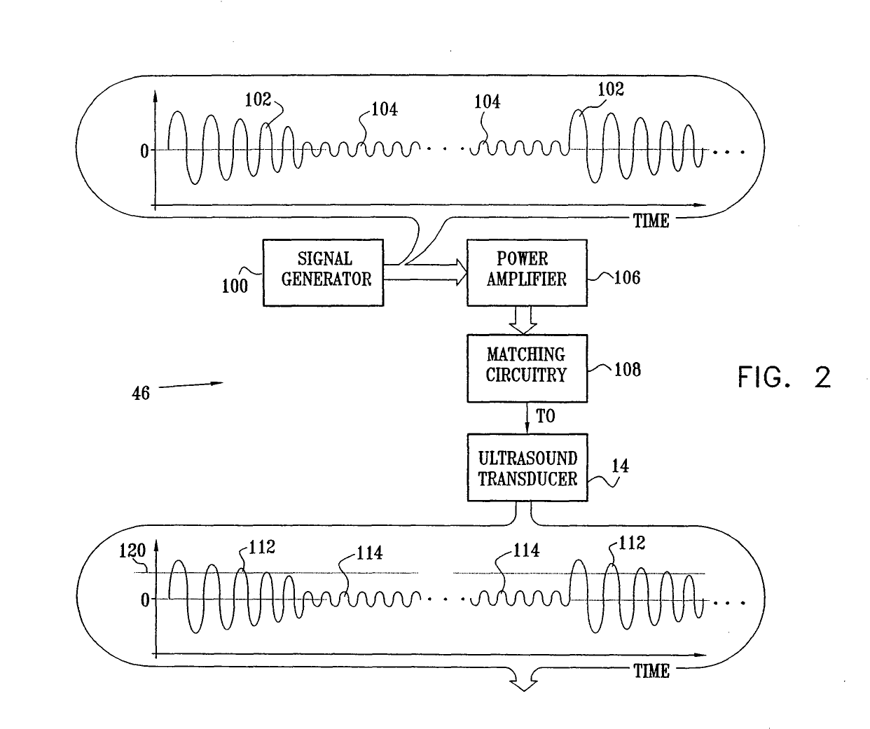

Ultrasound transducer subsystem 10 preferably receives suitably modulated electrical power from a power source and modulator assembly 46, forming part of a control subsystem 48. Control subsystem 48 also typically includes a termination of pregnancy control computer 50 and a display 52. A preferred embodiment of power source and modulator assembly 46 is illustrated in FIG. 2 and described hereinbelow. Ultrasound transducer subsystem 10 may be positioned automatically or semi-automatically as by an X-Y-Z positioning assembly (not shown). Preferably, ultrasound transducer subsystem 10 is positioned at a desired position by an operator.

FIG. 1 illustrates the transducer subsystem 10 being positioned on the body over a target volume 12 containing a gestational sac 54. Enlarged blocks designated by reference numerals 58 and 60 illustrate a typical target volume containing the gestational sac 54, respectively before and after termination of pregnancy in accordance with a preferred embodiment of the invention. It is seen from a comparison of blocks 58 and 60 that, in accordance with a preferred embodiment of the present invention, selective tissue destruction is presented within the target volume 12 containing gestational sac 54. The integrity of the gestational sac 54 is violated, while non-gestational sac tissue 62, such as portions of the uterus, for example, the basal layer of the endometrium and the myometrium, the ovaries, the cervix or any other intra or extra peritoneal organs, is not damaged.

Alternatively, the target volume may be selected to be smaller than the gestational sac. In such a case, selectivity of tissue destruction may prevent damage to non-gestational sac tissue in the event of incorrect location of the target volume.

Alternatively, selectivity of tissue destruction within the target volume may not be provided.

Reference is now FIG. 2, which is a simplified block diagram illustration of a preferred power source and modulator assembly 46 (FIG. 1), showing patterns of variation of ultrasound pressures over time in accordance with a preferred embodiment of the present invention employing cavitation. As seen in FIG. 2, the power source and modulator assembly 46 preferably comprises a signal generator 100 which provides time varying signals which are modulated so as to have a series of relatively high amplitude portions 102 separated in time by a series of typically relatively low amplitude portions 104. Each relatively high amplitude portion 102 preferably corresponds to a treatment period. Different signals generated by signal generator 100 may differ in phase as dictated by control subsystem 48 (FIG. 1) to achieve focus at a desired location.

Preferably the relationship between the time durations of portions 102 and portions 104 is such as to provide a duty cycle between 1:2 and 1:250, more preferably 1:5 and 1:100, and most preferably between 1:10 and 1:80. Alternatively, continuous, e.g. non-pulsed, ultrasound energy may be employed.

Preferably, the output of signal generator 100 has a frequency in a range of 100 KHz-5000 KHz. As the desired dimensions of the target volume are decreased, the frequency increases within the abovementioned range. Accordingly, if a target volume is employed which is smaller than the gestational sac, e.g. a target volume of approximately 0.5 cubic centimeters is employed, the frequency will preferably be between 1000 KHz-3000 KHz. Similarly, if a relatively large target volume, such as 2 cubic centimeters is employed, the frequency will preferably be between 100 KHz and 300 KHz.

The system of the present invention may be operative in various possible modes of operation, including, for example, cavitation, thermal, micro streaming and jackhammer.

When the system is operative to provide cavitation or micro streaming, the frequency is preferably between 100 KHz-1000 KHz and more preferably between 200 KHz and 700 KHz When the system is operative in a thermal mode of operation, the frequency is preferably 1 MHz-5 MHz. The foregoing frequencies may apply to both pulsed and continuous energy application.

The output of signal generator 100 is preferably provided to a suitable power amplifier 106, which outputs via impedance matching circuitry 108 to an input of ultrasound therapeutic transducer assembly 14 (FIG. 1), which converts the electrical signal received thereby to a corresponding ultrasound energy output. As seen in FIG. 2, the ultrasound energy output preferably comprises a time varying signal which is modulated correspondingly to the output of signal generator 100 so as to having a series of relatively high amplitude portions 112, which exceed an effective cavitation threshold 120 and which correspond to portions 102, separated in time by a series of typically relatively low amplitude portions 114, corresponding to portions 104.

Preferably, each high amplitude portion 112 comprises between 2 and 1000 sequential cycles at an amplitude above the cavitation threshold 120, more preferably between 25 and 500 sequential cycles at an amplitude above the cavitation maintaining threshold 120 and most preferably between 100 and 300 sequential cycles at an amplitude above cavitation threshold 120.

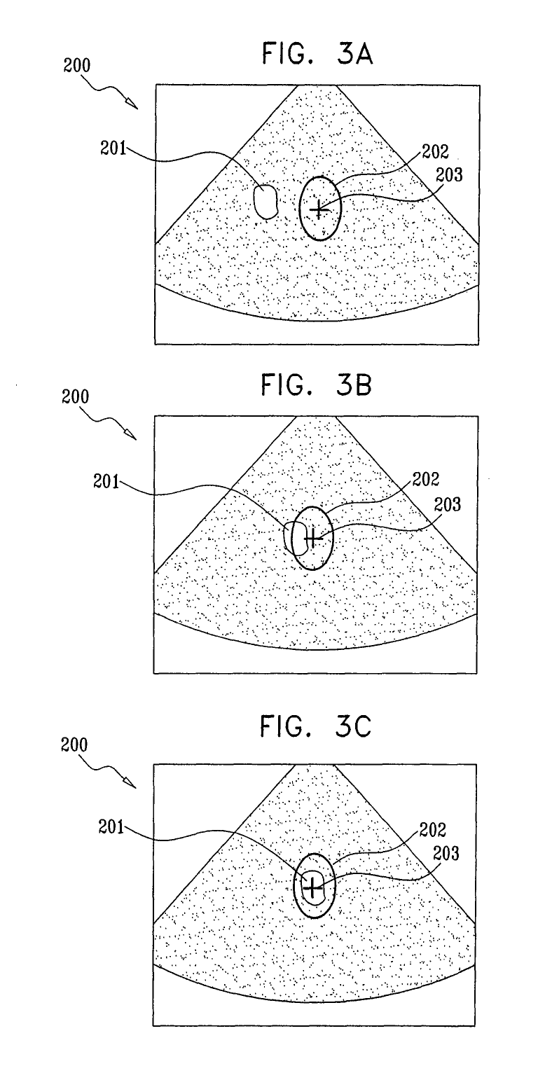

Reference is now made to FIGS. 3A, 3B and 3C, which are simplified pictorial illustrations of the appearance of an operator interface display during operation. As seen in FIG. 3A, during operation, display 52 (FIG. 1) typically shows an ultrasound B mode image 200 including an image of the gestational sac 201. Additionally, display 52 shows the location 202 of the target volume 12 (FIG. 1) and therewithin, the calculated focus 203 of the ultrasound energy beam. Repositioning of transducer subsystem 10 (FIG. 1) with respect to the body changes the relative position of the gestational sac 201 and calculated focus 203 of the energy beam as seen in FIG. 3B until they overlap, as seen in FIG. 3C.

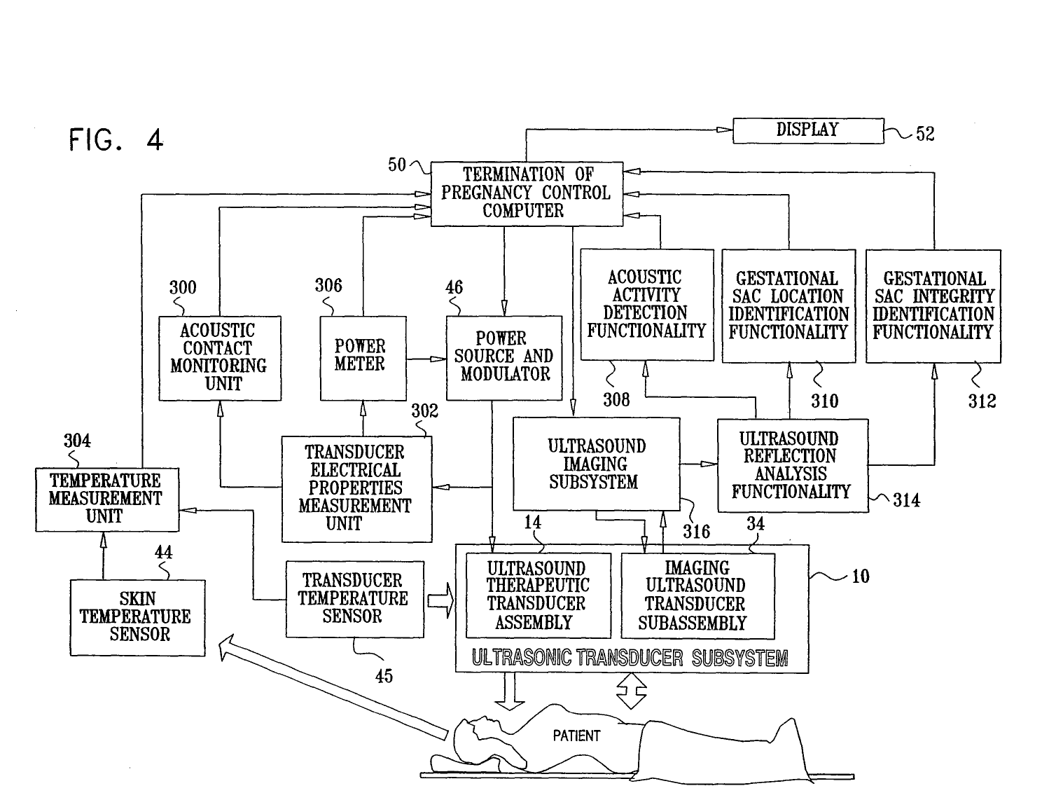

Reference is now made to FIG. 4, which illustrates a termination of pregnancy treatment system constructed and operative in accordance with a preferred embodiment of the present invention. As described hereinabove with reference to FIG. 1 and as seen in FIG. 4, the termination of pregnancy treatment system comprises a termination of pregnancy treatment control computer 50, which outputs to a display 52. Termination of pregnancy treatment control computer 50 preferably receives an input from an acoustic contact monitoring unit 300, which in turn preferably receives an input from a transducer electrical properties measurement unit 302.

Termination of pregnancy treatment control computer 50 also preferably receives an input from a temperature measurement unit 304, which receives temperature inputs from skin temperature sensor 44 (FIG. 1) and transducer temperature sensor 45 (FIG. 1). Temperature measurement unit 304 preferably compares the outputs of both sensors 44 and 45 with appropriate threshold settings and provides an indication to termination of pregnancy treatment control computer 50 of threshold exceedence. Transducer electrical properties measurement unit 302 preferably monitors the output of power source and modulator assembly 46 (FIG. 1) to ultrasound therapeutic transducer assembly 14.

An output of transducer electrical properties measurement unit 302 is preferably also supplied to a power meter 306, which provides an output to the termination of pregnancy treatment control computer 50 and a feedback output to power source and modulator assembly 46.

Termination of pregnancy treatment control computer 50 also preferably receives inputs from acoustic activity detection functionality 308, gestational sac location identification functionality 310 and gestational sac integrity identification functionality 312, all of which receive inputs from ultrasound reflection analysis functionality 314. Ultrasound reflection analysis functionality 314 receives ultrasound imaging inputs from an ultrasound imaging subsystem 316, which operates imaging ultrasound transducer subassembly 34 (FIG. 1).

Termination of pregnancy treatment control computer 50 provides outputs to power source and modulator assembly 46, for operating ultrasound therapeutic transducer assembly 14, and to ultrasound imaging subsystem 316, for operating ultrasound imaging transducer subassembly 34. Optionally, a positioning control unit (not shown) may be provided and receive an output from termination of pregnancy treatment control computer 50 for driving an X-Y-Z positioning assembly (not shown) in order to correctly position transducer subsystem 10.

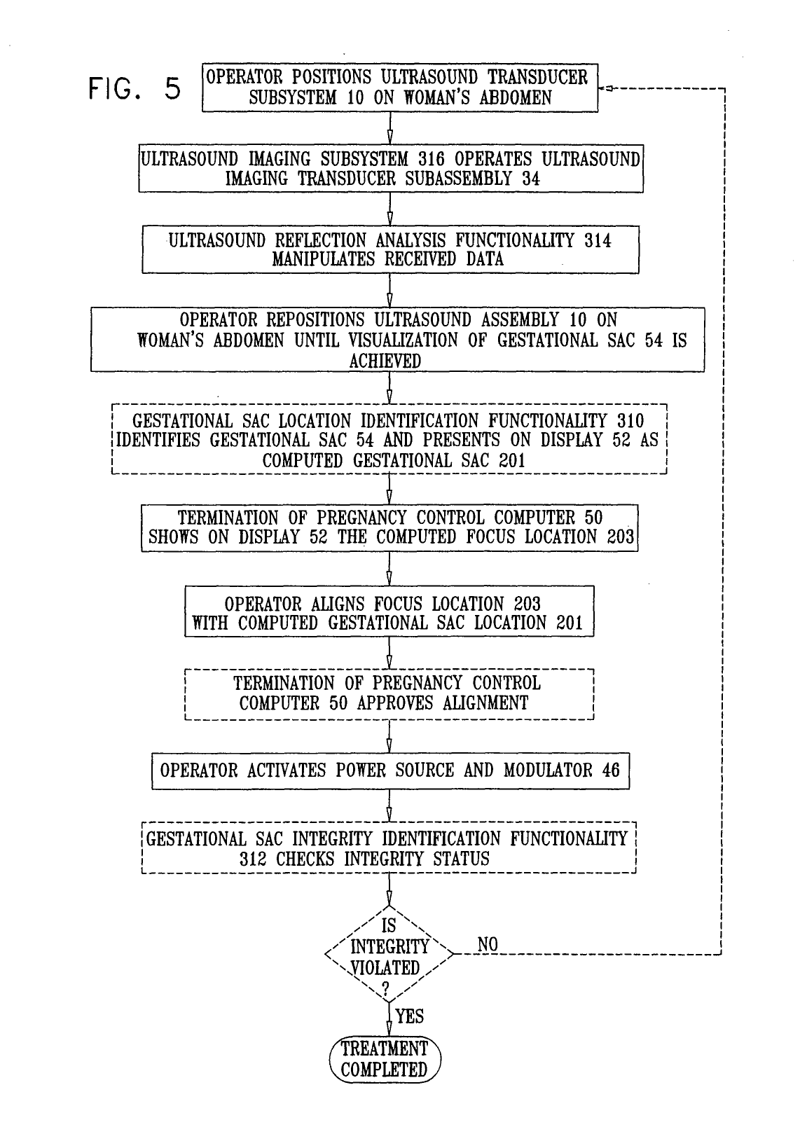

Reference is now made to FIG. 5, which is a simplified flowchart illustrating operator steps in carrying out termination of pregnancy treatment in accordance with a preferred embodiment of the present invention. As seen in FIG. 5, initially an operator preferably positions transducer subsystem 10 on a woman's abdomen. The ultrasound imaging subsystem 316 (FIG. 4) operates ultrasound imaging transducer subassembly 34, causing it to provide an output to ultrasound reflection analysis functionality 314 for analysis.

Ultrasound reflection analysis functionality 314 manipulates received data and presents it on display 52 enabling the operator to position ultrasound assembly 10 on the women's abdomen at a location where visualization of the gestational sac is achieved. Gestational sac location identification functionality 310 (FIG. 4) is preferably provided to identify gestational sac 54, although this function may be performed by a human operator.

Using the visualization, the operator adjusts the location 202 (FIG. 3) of the target volume 12 and therewithin, the calculated focus 203 of the ultrasound energy beam. Repositioning of transducer subsystem 10 with respect to the body proceeds until the location 202 with focus location 203 at its center, overlaps the location of the gestational sac 201, as seen in FIG. 3C. The repositioning may be effected manually by the operator, mechanically or electronically.

Preferably an operator, and alternatively termination of pregnancy control computer 50, approves the positioning of the transducer subsystem 10. Thereafter the operator activates power source and modulator 46 preferably according to preset parameters as defined by the termination of pregnancy computer 50, thereby applying ultrasound energy to the target volume including at least part of the gestational sac, thereby achieving termination of pregnancy.

Optionally, during application of ultrasound energy to the target volume, acoustic activity detection functionality 308 may provide confirmation of impingement of the ultrasound on the target volume. Functionality 308 may alternatively or additionally receive real time imaging outputs from ultrasound imaging transducer subassembly 34 via ultrasound image subsystem 316, which confirm and indicate the location of the acoustic activity at the target volume.

Following application of ultrasound energy to the target volume, optionally, gestational sac integrity identification functionality 312 may provide visual confirmation that the integrity of the gestational sac has been violated.

It will be appreciated by persons skilled in the art that the present invention is not limited by what has been particularly shown and described hereinabove. Rather the scope of the present invention includes both combinations and subcombinations of various features described hereinabove as well as modifications and variations thereof which may occur to a person skilled in the art upon reading the foregoing description and which are not in the prior art.

1-33. (canceled)

34. Apparatus for termination of pregnancy comprising:
a radiant energy source; and
a gestational sac irradiator, adapted to be operative to irradiate a gestational sac through non-gestational sac body tissue with sufficient energy from said radiant energy source so as to cause termination of pregnancy.

35. Apparatus for termination of pregnancy according to claim 34 and also comprising a gestational sac imager.

36. Apparatus for termination of pregnancy according to claim 34 and also comprising a radiant energy modulator operative to modulate said radiant energy so as to have characteristics which enable termination of pregnancy without substantial pathological effects on non-gestational sac body tissue within an irradiated target volume containing said gestational sac.

37. Apparatus for termination of pregnancy according to claim 34 were the radiant energy is focused energy.

38. Apparatus for termination of pregnancy according to claim 35 wherein said imager provides ultrasound imaging.

39. Apparatus for termination of pregnancy according to claim 35 wherein said imager provides MRI imaging.

40. Apparatus for termination of pregnancy according to claim 35 wherein said imager provides CT imaging.

41. Apparatus for termination of pregnancy according to claim 34 wherein said energy comprises ultrasound energy.

42. Apparatus for termination of pregnancy according to claim 34 wherein said energy comprises electromagnetic energy.

43. Apparatus for termination of pregnancy according to claim 34 wherein said gestational sac irradiator produces a thermal effect on the gestational sac.

44. Apparatus for termination of pregnancy according to claim 41 wherein said gestational sac irradiator produces a cavitation effect on the gestational sac.

45. Apparatus for termination of pregnancy according to claim 41 wherein said gestational sac irradiator produces a micro-streaming effect on the gestational sac.

46. Apparatus for termination of pregnancy according to claim 41 wherein said gestational sac irradiator produces a jackhammer effect on the gestational sac.

47. Apparatus for termination of pregnancy according to claim 35 where said imager is operative to image results of operation of said gestational sac irradiator.

48. Apparatus for termination of pregnancy according to claim 34 and wherein said gestational sac irradiator includes an energy director operative to direct said energy to a target volume at least partially including the gestational sac.

49. Apparatus for termination of pregnancy according to claim 48 wherein said director generally prevents pathological damage to tissue outside of said target volume.

50. Apparatus for termination of pregnancy according to claim 48 wherein said director generally focuses the energy on the gestational sac.

51. Apparatus for termination of pregnancy according to claim 48 wherein said director comprises at least one transducer selectably positionable relative to the body.

52. Apparatus for termination of pregnancy according to claim 48 wherein said director focuses energy from at least one transducer at said target volume.

53. Apparatus for termination of pregnancy according to claim 48 wherein said director is operative to vary a location of a focus of at least one transducer at said target volume.

54. Apparatus for termination of pregnancy according to claim 53 and wherein said director, by varying said location of said focus, changes a volume of said target volume.

55. Apparatus for termination of pregnancy according to claim 34 and also comprising an irradiation feedback indication functionality.

56. Apparatus according to claim 51 and wherein said transducer is adapted to be located outside of the body.

57. Apparatus according to claim 41 and wherein said ultrasound energy has a frequency in a range of 100 KHz-5000 KHz.

58. Apparatus according to claim 41 and wherein said ultrasound energy has a frequency in a range of 100 KHz-300 KHz.

59. Apparatus according to claim 41 and wherein said ultrasound energy has a frequency in a range of 1000 KHz-3000 KHz.

60. Apparatus for termination of pregnancy according to claim 48 including an energy modulator to effect termination of pregnancy while generally preventing pathological damage to non-gestational sac tissue within said target volume.

61. Apparatus for termination of pregnancy according to claim 60 wherein said energy is ultrasound energy.

62. Apparatus for termination of pregnancy according to claim 61 and wherein said modulator provides a duty cycle between 1:2 and 1:250.

63. Apparatus for termination of pregnancy according to claim 61 and wherein said modulator provides a duty cycle between 1:5 and 1:100.

64. Apparatus for termination of pregnancy according to claim 61 and wherein said modulator provides a duty cycle between 1:10 and 1:80.

65. Apparatus for termination of pregnancy according to claim 61 and wherein said modulator provides between 2 and 1000 sequential cycles at an amplitude above a cavitation threshold.

66. Apparatus for termination of pregnancy according to claim 61 and wherein said modulator provides between 25 and 500 sequential cycles at an amplitude above a cavitation threshold.

67. Apparatus for termination of pregnancy according to claim 61 and wherein said modulator provides between 100 and 300 sequential cycles at an amplitude above a cavitation threshold.

68. Apparatus for termination of pregnancy according to claim 61 and wherein said modulator modulates the amplitude of said ultrasound energy over time.

69. Apparatus for termination of pregnancy according to claim 41 and wherein said irradiator employs ultrasound energy in a continuous mode.
